(19) United States
(12) Patent Application Publication
(10) Pub. No.: US 2004/0133358 A1
(43) Pub. Date: Jul. 8, 2004

(54) Clinical and diagnostic database and related methods

(76) Inventors: Stephen Paul Bryant, Cambridge (GB); Paul James Kelly, Cambridge (GB); Peter Wayne Reed, Cambridgeshire (GB)

Correspondence Address: MORRISON & FOERSTER LLP, 3811 VALLEY CENTRE DRIVE, SUITE 500, SAN DIEGO, CA, 92130-2332, US

(21) Appl. No.: 10/740,043

(22) Filed: Dec. 17, 2003

Related U.S. Application Data
(63) Continuation of application No. 09/514,955, filed on Feb. 28, 2000, now abandoned.

(30) Foreign Application Priority Data
Feb. 26, 1999 (GB) 9904585.8

(51) Int. Cl. G06F 17/60; G06F 19/00; G01N 33/48; G01N 33/50

(52) U.S. Cl. 702/19; 702/20; 705/3

(57) ABSTRACT

A clinical and diagnostic database comprises a plurality of records which each contain phenotype information and optionally sample information for an individual. The record for the individual further comprises confounding information, and the sample information for the individual comprises information relating to the location of a sample of tissue or of fluid from the individual. The confounding information is taken into account in generation of correlations between phenotypes and genotypes.

### DESCRIPTION OF BACKGROUND ART

1. Field of the Invention

The present invention relates to a database containing information useful for clinical, diagnostic and other purposes, and relates in particular to a database containing genotype and phenotype information. The present invention also relates to methods of adding information to the database and to methods of identifying correlations within and between phenotypes and/or genotypes in the database as well as to other uses of the database.

2. Related Art

It is recognised that most diseases can be correlated with geographical, environment, dietary, genetic and/or other specific contributory factors. Hence, much effort today is directed at identifying those contributing factors, and also those factors which may not directly contribute to these but are otherwise linked thereto and may be correlated with presence of disease for some other reason, so that even more accurate diagnosis of disease and pre-deposition to disease can be achieved.

It is known to select a group of individuals according to a particular criteria and carry out various tests including obtaining information such as genotype and phenotype to create a database of information concerning individuals conforming with the particular selection criteria chosen. Information in the database may then be used to identify causative factors or other factors related to incidence of or pre-deposition to disease. Following this strategy, it is known, for example, to carry out an analysis of the causes of the hypertension by selecting a group of individuals all of which are hypertensive and then attempting to identify common genotypic or phenotypic characteristics amongst this group. When analysing the causes of the different disease, a different selected group of individuals is identified and may be subject of a separate analysis.

### DETAILED DESCRIPTION OF THE INVENTION

The present invention relates to a database and methods of maintaining the database and methods of use thereof which represents a new approach to obtaining correlations between phenotype and genotype as well as cross-correlation between phenotypes, and cross correlations between phenotypes and genotypes.

It is an object of the present invention to provide a database containing phenotype and also preferably genotype information that can readily be used to obtain clinically and/or therapeutically and/or diagnostically useful information. A further aim is to provide a database of phenotype and also preferably genotype information which can readily be updated and expanded and adapted according to a wide ranges of uses proposed for the data within the database. A still further object of the present invention is to provide methods of obtaining clinically or therapeutically or diagnostically useful information from the data stored in the database of the present invention.

According to a first aspect of the invention there is provided a database comprising a plurality of records, said records containing phenotype information and optionally sample information for an individual, wherein the record for the individual further comprises confounding information, and the sample information for the individual comprises information relating to the location of a sample of tissue or of fluid from the individual.

The invention confers the advantage that by using the stored records it is possible to identify disease or potential disease or risk of disease in people who do not yet have any signs of disease or at least have no significant outward signs of disease. Preferably records for the database are obtained and then can optionally be updated, for example by retesting, from such individuals who do not yet have any signs of disease or at least have no significant outward signs of disease.

Thus according to the invention, a phenotype can be identified as being measurable in most or all people, it is then measured and the database enables identification of genes that influence risk; where it is known how risk factors affect disease the database can be used to determine how a gene can affect risk factors.

For example, using the database it is possible to identify, say, a group of individuals who possess a given genotype, that is to say a given form of a gene, which influences blood pressure; as it is known how blood pressure influences disease, say coronary heart disease, an assessment of risk of this disease can be calculated for that gene.

Suitably, the record for an individual comprises information relating to a plurality of phenotypes and the record comprises, in respect of each phenotype:—

the phenotype observed; and

information relating to actual or potential confounding indicators in respect of phenotype.

The confounding information enables phenotypes that are influenced by that type of confounding information to be adjusted or otherwise labelled accordingly. As an example, knowledge that an individual is a smoker is relevant to trying to correlate airways disease to a genetic cause, as airways disease will also be affected by smoking.

The invention thus offers the advantage that account may be taken of the confounding factors and more reliable correlations obtained from the records in the database.

A number of different types of confounding information are of relevance to the database of the invention. By way of example, the database can optionally contain confounding information selected from the group consisting of medication being taken by the individual, medical history, occupational information, information relating to the hobbies of the individual, diet information, family history, normal exercise routines of the individual, age and sex. More specific examples of confounding information include whether the individual is undergoing hormone replacement therapy, is the individual a drinker, is the individual a smoker, does that person regularly use a sunbed, where geographically does that person reside, how much exercise does that person take, is the individual post or pre-menopausal. Preferably, the phenotype and confounding information is collected at the same time from the individual, so that the confounding information is of the most relevance to the phenotype.

More specifically, a database of the invention comprises a plurality of records, each record containing phenotype information, and optionally sample information, for an individual, wherein:

the phenotype information for the individual comprises at least one of and preferably all of osteoporosis related phenotypes, osteoarthritis related phenotypes, immune cell subtypes (such as T cell subsets), metabolic syndrome/syndrome X related phenotypes, and hypertension related phenotypes; and

the sample information for the individual comprises information relating to the location of a sample of tissue or of fluid from the individual.

The database is suitable for storage of records relating to a wide variety of different individuals, and is especially suitable for information relating to human individuals though it is equally suited for use with animal or other veterinary data, preferably mammalian data. The inclusion of sample information in the database enables users of the database to locate a sample of tissue or of fluid from the individual for further testing. This further testing might be to obtain additional phenotype information not previously tested from that tissue or fluid sample or it might be to confirm and possibly correct or update phenotype data already stored for a particular characteristic of that individual. The database is also suitable for correlation with other proprietary and public databases consisting of clinical information, data on genomics, proteonomics, cell biology, immunology and biochemistry. Furthermore the database is interactive and allows cross correlation of key genotypes/haplotypes with key phenotypes to better understand the biology, and regulation of genetic, cell biological and humoral networks involved in complex diseases.

A further advantage of the invention is that it is possible to go back to a given group of people who have records in the database and test or retest in respect of a given disease, and this is facilitated by the inclusion of sample information.

The type of tissue or fluid samples that can be stored in accordance with the invention are without limits. Typically, fluid samples that can readily be stored include urine, serum and saliva samples. Tissue samples that can readily be stored include skin, liver, heart tissue, bone, hair, muscle, kidney, tooth and faeces samples. Most of these tissue or fluid samples will contain DNA. Nevertheless, it is also an option for a separate sample to be stored containing DNA extracted from tissue of that individual. To enable easy location of the tissue of the fluid sample it is typical for the sample information to include the geographical location of the sample, for example the address of the storage institution, as well as the storage conditions and the storage reference number or storage identification number to enable identification and retrieval of the sample when needed.

Records in the database are preferred also to contain genotype information relating to the individual, such as one or more single nucleotide polymorphisms (“SNPs”) in the DNA of the individual. Alternatively or additionally, the genotype information can comprise a record of actual or inferred DNA base sequence at one or more regions within the genome. Still further, the genotype information can comprise a record of variation between a specified sequence on a chromosome of that individual compared to a reference sequence; indicating whether and to what extent there is variation at identical positions within the sequence. The genotype information can yet further comprise a record of the length of a particular sequence or a particular sequence variant; such information being of use to investigate absence or presence of correlation between genetic variation and phenotype variation.

In this and related contexts, reference to genotype is intended to refer to genotype or to haplotype or to both genotype and haplotype. In use of an example of the invention, SNPs from proprietary or public domain databases are added to and stored in the present database for the individuals. It is then possible to try to identify an association between one or more of these SNPs by correlation with one or more phenotypes stored in the present database. One method to achieve this is to search the DNA of an individual for one or more polymorphisms which are associated with a given risk trait, the polymorphisms being for example SNPs with allele frequencies of at least 20%, and which do not have linkage disequilibrium.

It is preferred that a large amount of phenotype information is recorded in the database for each individual, and also preferred that all or substantially all of this information is obtained via a single interview and/or examination or if necessary via numerous such sessions over a short time frame. The types of phenotypes stored can usefully include quantitative risk traits associated with chronic diseases, biochemical parameters, cell biological parameters such as cell surface markers and factors of cell growth, apoptosis and signal transduction. structural and humoral proteins and other biochemicals and metabolites.

In a preferred embodiment of the invention, the phenotype information recorded further includes thrombosis/fibrinolysis phenotypes, haemoglobinopathy related phenotypes and airways disease (asthma) phenotype. In this and related contexts, reference to phenotypes is intended to be a reference to data relating to at least one phenotype and typically more than one phenotype of the nature indicated. Additional phenotype information used in still further preferred embodiments of the invention relates to the phenotypes: atopy/eczema, lung function, IgE, psoriasis, acne, skin cancer and moliness of skin.

Other information that may be included in the category of phenotype information that can be included in the database comprises information relating to quantitative traits related to cognition, dementia, Parkinson's disease and intelligence, history of adverse drug reactions and history of substance abuse/addictive behaviour.

It is thus apparent that the database of the invention may hold information on phenotypes in a hitherto unmatched number of categories. This extensive breadth of information in specific embodiments of the invention contributes to the uniquely valuable information that can be extracted therefrom in the various applications of the database described below.

Still further optional areas of phenotype information that are include in the database relate to: lifestyle—such as alcohol, tobacco, diet, exercise—, dietary history, medication history and family history of disease.

The sample information may additionally include contact information so as to enable the individual whose data is already in the database to be contacted and recalled for further testing.

It is an advantage of having the sample information that data in the database can be checked, corrected and/or expanded by further testing of the tissue or fluid samples that have been stored for each individual. In the case of an unusual value being recorded for a particular phenotypic characteristic, a tissue or fluid sample can be retested to confirm the information in the database. Whilst it is believed that the phenotype stored in the database will be sufficient to enable a wide range of uses of the data, it is envisaged that some particular investigations will call for phenotype information that has not yet been tested for individuals in the database, or has not been tested in the manner required for a particular investigation. In these circumstances it is particularly advantageous that the tissue or fluid sample can be recalled and tested to add in the required additional phenotype information to that phenotype information already present in the database. The further testing of stored material in this way is considerably more convenient and efficient than trying to locate individuals that have been included in the database and arrange for further testing of missing phenotype information in person.

In a database of the invention, phenotypic data are generally maintained for each individual within the database with most data being associated not only with an individual, but also with a particular timepoint. Some physiological results vary over time and are valid in relation to each other only if collected at the same timepoint.

Stored material (DNA, Serum and Urine) is preferably maintained for each individual, for each visit. Additional phenotype data may be collected by performing assays on stored material, which will not deteriorate appreciably, even over several years. There is therefore the potential to expand the phenotype within the database of the invention, even if the assays are not carried out at the time of the visit. It is also possible to expand the phenotype by conducting questionnaires, interviews or other measurements, if the results are not expected to vary over time, or else vary predictably. This can include a) historical medical data, b) family history and c) drug usage.

There is also the option of collecting longitudinal data by having the individual return for a repeat visit. In this case, all the time-sensitive results are distinctly recorded within the database, which permits another dimension of analysis (time, or ageing) to be carried out. Some measurements from repeat visits would not necessarily be time-dependent and could be analyzed against results collected at earlier visits. Also, new technologies are brought in from time to time and can be used to “top-up” the phenotype. For straightforward analyses of a single outcome phenotype against the genetic background (which does not vary over time), it does not matter that these additional phenotypes are collected over a period of years, and this method is validly used to expand the database phenotype by a managed programme of revisits.

In a further embodiment of the invention, there is provided a method of integrating (a) information either in the private or public domain on genomic, proteonomics, cell and molecular biology and /or immunology with (b) information on the database of the invention, which information is collected on the patient population, and determining if there are any correlations between them.

In a second aspect of the invention, there is provided a method of adding information to the database of the invention, comprising:

1. identifying an individual not yet included in the database;

determining phenotype information for the individual that comprises at least osteoporosis related phenotypes, osteoarthritis related phenotypes, immune cell subtypes (such as T cell subsets), metabolic syndrome/syndrome X related phenotypes, and hypertension related phenotypes;

optionally determining genotype information for that individual;

optionally determining sample information for the individual that includes information relating to the location of a sample of tissue or of fluid from the individual; and

creating a record in the database to hold the phenotype and optionally genotype and/or sample information for the individual; or

2. identifying an individual already included in a record in the database;

using sample information in the database to obtain a tissue or fluid sample for the individual;

testing the sample, thereby determining genotype or phenotype information for the individual; and

adding or confirming or amending or updating information in the record for the individual.

The method of the second aspect of the invention represents improvement over the operation of prior art databases, in that the information stored in the database of the present invention can continually and without limit be expanded and updated and, if need be, corrected. The information in the database of the present invention does not reach a point at which it needs to be discarded and a new database started. Instead, the information can be obtained and amassed in a cumulative way so that the database is forever becoming more useful and more accurate for obtaining clinically or therapeutically or diagnostically useful information. It is particularly preferred that the information stored in the database of the invention is obtained from individuals who have not been selected according to any particular genotype and/or phenotype characteristic. That is to say, whereas in the prior art a cohort of individuals might have been selected for use in a genotype and phenotype database because they all had low bone mineral densities, the individuals included in the database of the present invention are not selected in this way. Instead, genotype and phenotype information from all and any individuals may be included in the database. Thus, taking the latter example of bone mineral density, the phenotype of bone mineral density is selected and then all individuals are tested and the results recorded. It is not required that all have, say, low scores. The phenotype is tested and no individuals are selected according to their characteristics in respect of that phenotype. Particularly preferred is that twins are included in the database having different confounding information in respect of a selected phenotype.

A disadvantage of prior art databases was that the cohort of individuals selected, for example, for an investigation into bone mineral density and the factors affecting bone mineral density would not be suitable for a separate investigation into, say, the effect of diet on blood pressure. The database of the present invention does not suffer from this disadvantage because the individuals in the database of the present invention have not been selected with any one particular clinical investigation in mind and are advantageously suitable for use in substantially all such investigations.

Further aspects of the invention relate to uses of the information contained in the database of the invention. Accordingly, a third aspect of the invention provides a method of identifying a correlation between phenotype information and genotype information comprising:

selecting a phenotype characteristic;

identifying a plurality of records from the database of the invention for individuals that comply with the selected phenotype characteristic;

determining if presence of the selected phenotype characteristic is correlated with presence of any genotype characteristic in the genotype information for records in the database.

A fourth aspect of the invention provides a method of identifying a correlation between phenotype information and phenotype information comprising:

selecting a phenotype characteristic;

identifying a plurality of records in the database for individuals who comply with the phenotype characteristic;

determining if presence of the selected phenotype characteristic is correlated with another characteristic of phenotype information for records in the database.

More specifically, the method can comprise identifying correlation between presence of the selected phenotype characteristic and two or more separate characteristics of phenotype information for records in the database A fifth aspect of the invention provides a method of identifying a correlation between genotype information and genotype information comprising:

selecting a genotype characteristic;

identifying a plurality of records in the database for individuals who comply with the genotype characteristic;

determining if presence of the selected genotype characteristic is correlated with another characteristic of genotype information or records in the database.

In use of the invention, there is provided a method of allocating priority to a candidate gene or locus, proposed as a drug target for treatment of a disease, the method comprising:—

calculating, from data on a database according to the invention, the specificity of the candidate gene or locus for the disease;

comparing (i) the association of the disease with clinical risk traits related to the disease, to (ii) the association of the disease with other clinical risk traits unrelated to the disease, but representing significant side effects; and

hence calculating a likely therapeutic index of drug candidates acting on that gene or locus.

For a top priority gene, the information on the database is used for correlating genotype with clinical risk traits, and with associated biochemical and cell biology phenotypes. This can give valuable information on the targets and mechanisms of action, and the biochemical pathways.

In a further general use of the invention, there is provided a method of analysing the relation between a genotype and a phenotype, comprising

selecting a phenotype characteristic;

identifying a plurality of records complying with that characteristic;

using environmental and age-related data in the database to eliminate the effects of age and environment on variations in phenotype; and

hence calculating from the database whether and if so to what extent the phenotype is correlated with a particular genotype.

In a further example of the invention in use, there is provided a method of determining the capacity and specificity of a genetic marker to detect and quantify normal variations in healthy and affected populations for a selected risk trait, comprising:—

assaying a sample in the database for the marker levels, in both healthy and affected subjects; and

quantifying the association of the clinical trait with the marker level and other selected phenotypes, in unaffected and affected subjects.

Another use of the invention lies in a method of predicting the response of patients to a selected drug therapy in a clinical trial, comprising:—

selecting a proposed clinical population for the trial;

using data on the database to stratify the clinical population by high associations of metabolism/absorption both with genotype and/or with associated biochemical and cell biology phenotypes; and

hence allowing definition of the best dose regimes and dose forms/drug delivery systems;

so as to predict and/or allow for absorption and/or metabolism of the drug by patients in the clinical population.

A yet further example of the invention in use provides a method of predicting response to a proposed drug therapy, comprising:—

using the database to select a clinical population by constructing haplotypic profiles, with strong associations with defined clinical traits and biochemical phenotypes;

using the database, and the twin resource, to eliminate the effects of age and environment in the clinical population;

hence providing criteria to predict response to the drug and variation in response to the drug, and optionally to define a sub-group of the clinical population or of the general population most susceptible to the drug being studied.

Twins are useful for controlling quantification of the impact of environmental factors on disease risk and are suitable for inclusion in a database of the invention. Identical twins share the same genes so any difference in a clinical measurement within an identical twin pair must be due to environmental factors or measurement error. By studying sufficient numbers of identical twins and measuring relevant environmental factors one can quantitate the impact of the environmental on clinical measurements.

Also, twins can be identified who are discordant for an environmental exposure. For example by examining fat mass where one twin from sufficient numbers of subjects where one identical twin of a pair smokes and the other does not one can quantitate the impact of smoking on obesity (Samaras et al Int J Obesity 1998). This can be made more sophisticated by doing such an analysis in twins who are concordant or discordant for other environmental factors, for instance exercise level. If the quantitative impact of various environmental factors is also known then one should be able to integrate that information into a multivariate model, along with candidate gene or candidate loci data, to identify gene-environment interactions.

Twins are followed prospectively and have further phenotypic data collected and also further DNA, serum, urine or tissue samples collected.

Samples taken from twins at any one clinical visit are stored to be used at any future. These can be reanalysed for new biochemical or serological analytes and related to historical clinical and genetic data. Moreover, DNA is stored and can be retrieved for further genetic analysis as required. Lymphocytes cells are frozen and stored for future immortalisation to allow an ‘infinite’ DNA resource.

Phenotypes relating to many clinical diseases (either their presence or absence or the risk of these diseases) in the twins novel correlations between phenotypes can be identified that could not be so if the data collection was solely focused on a more limited phenotype set. This is carried out by various forms of correlational and cluster analysis to identify novel relationships between quantitative traits relating to broad disease areas. For instance relating phenotypes in anxiety and depression to those involved in diseases such as diabetes, osteoporosis, immunity, coagulation, may identify novel new disease entities that will be useful for clinical diagnosis;

design of clinical trials;

targeted therapeutic intervention;

identification of new disease targets for drug discovery;

identification and validation of new molecular targets for drug discovery programmes; and

identification of patient populations most susceptible to chronic illnesses and hence to therapy.

A clinical and diagnostic database of the invention is of use in yielding disease-associated genes to form the basis of a drug discovery programme, the disease-associated gene being a gene for which novel clinical involvement is demonstrated. This association implies that a gene-based diagnostic or therapeutic could be developed to interfere with the functioning of the gene product. The identification of disease involvement further opens up the possibility of “rational drug design”, an approach that the industry regards as the basis of many future drugs. The association of a particular gene with a clinical risk trait for a common, age-related, chronic disease yields a suite of protectable claims, specifically:

the treatment (of several) diseases by administration of a modulator of the gene

Identification of compounds that modulate the gene (and which would be useful as therapeutic agents);

the diagnosis of disease or predisposition to disease by genotyping the gene; and/or diagnosis of disease based on one or more specific polymorphisms at specified positions A disease susceptibility is suitably delivered as a comprehensive clinical risk trait association report (between the genetic and phenotypic data in the clinical and diagnostic database).

The clinical samples and data are accessed to add value to existing candidate targets. By identifying polymorphisms (usually SNPs) in clinical populations that are part of the database of the invention and assessing the relevance to disease, the following discoveries and/or claims to further or related inventions may be made following a positive association:

each SNP in the gene could be used as part of a diagnostic assay;

the gene product itself as biotherapeutic or small molecule target; and/or

potential pharmacogenetic applications (e.g. patient profiling in clinical trials).

The database can also be used to discover disease-associated protein targets. By using high-throughput methods, e.g. 2D Gel Electrophoresis on serum samples from identical twins, it is possible to identify proteins that are susceptible to environmental influences, and which are associated with particular risk factors. These yield a pipeline of druggable targets directly, without requiring any positional cloning programme, since the proteins can be identified using mass spectrometry technology with no DNA analysis.

In use of the invention, a substantial genome scan has been completed on the database, consisting of 450 DNA markers on over two thousand non-identical twins. In total, 160 quantitative traits were analysed across several disease areas, including:

obesity/diabetes: fat mass, % and distribution, fasting insulin and glucose, triglycerides, leptin.

bone disease: ultrasound, BMD, BMC, bone turnover markers, hip spacing, vitamin D metabolites and binding protein.

cardiovascular: blood pressure, lipoproteins, coagulation factors, serum biochemistry.

immunology: T-cell antigens.

This programme yielded:

more than 100 chromosomal regions likely to contain genes involved in high market potential therapeutic areas; and

more than 50 regions taken forward into fine mapping and association studies.

The regions include two associated with osteoporosis and metabolic syndrome, and are further described below in specific embodiments of the invention.

The invention is further of use in discovery of novel disease/gene relationships. Specific embodiments of the invention, descibed in more detail below illustrate the capacity to:

rediscover genes with known disease involvement; and

identify novel associations with known genes.

There now follows description of specific embodiments of the invention for the purpose of non-limiting exemplification thereof.

### EXAMPLE 1

### Making a New Entry or an Addition to the Database

1. Initial Telephone Interview

The below-described protocol is followed to make a new entry in the database or to make an addition (or other change) to existing data.

The first stage is a telephone interview with one twin to request the following information:

Date of Birth

Address

Sex

Menopausal Status

Zygosity

Any serious illness or clinical conditions

How the interviewee heard about the study

Why the interviewee wishes to participate

The responses are recorded in an administration database and are used when calling subjects for interview as and when required.

2. Arrangements for the Study Day

Any individual who has had the initial interview may be called. Alternatively, as and when requirements for particular kinds of twin arises (e.g. sex, age) the database is interrogated and details of twins with the relevant profile are flagged out of the system—the example is thus written for the case that twin data is being added, though the same protocol is used for non twin data.

3. The Study Day

The following routine tests are carried out on each twin:

Fasting blood tests

Urine Tests

Anthropometric Measurements

Blood Pressure

Arterial Distensibility

DEXA Scanning: bone density and body composition

Muscle Strength: leg extensor power rig

Heel Ultrasound Scan

Spirometry

Electrocardiography

MRI Scans

X-Rays

Occasionally, other tests will be added for a particular study. A checklist is compiled for each test, completed as the interview progresses.

Questionnaires are also administered to the twins. Some during the study day, some which are sent out with the appointment letter and others provided as “homework” to complete after the visit day for sending in to the unit at a later date. The questionnaires contain a large number of questions on family history, medical history, current status and physical findings. Prospective questionnaires are required on certain clinical topics. In such cases, twins are given questionnaires to complete at home after the visit.

4. Processing Blood and Urine Samples

The following samples are taken:

Time 0 Glucose Tolerance Test (GTT)

30 ml clotted sample (3×10 ml tubes brown top)

40 ml EDTA (4×10 ml purple top)

2 ml fluoride/oxalate tube (grey top)

Time 120 after GTT (if done)

10 ml clotted sample (1×10 ml plain tubes brown top)

2 ml fluoride/oxalate tube (grey top)

4.1 Clotted Samples

Clotted samples for serum are spun at 3000 rpm in a suitable centrifuge for 10 minutes after standing for 2-4 hours.

a. Time 0 samples:

1×500 microlitre sample for routine biochemistry

12×1.5 ml cryotubes with green tops (approximately 750 microlitres/tube)

1×300 microlitre sample for sex hormones (as requested)

b. Time 120 samples:

4×1.5 ml cryotubes with green tops (approximately 750 microlitres/tube)

4.2 EDTA samples

These samples are for DNA extraction.

a) the sample is spun at 3,000 rpm for 10 minutes in a clinical centrifuge;

b) the buffy coat (the leucocytes, a yellowish layer of cells on top of red blood cells) is removed and pooled into a 15 ml conical tube;

c) 0.9% saline is added to fill the tube and resuspend the leucocytes. If there is a time delay, the sample can be stored at 4° C. for up to 48 hours;

d) the sample is spun at 2,500 rpm for 10 minutes at 4° C.;

e) the buffy coat is again removed as cleanly as possible leaving behind any red cells, the sample is suspended in cold red cell lysis buffer and left for 20 minutes at 4° C.;

f) the sample is spun again at 2,500 rpm for 10 minutes. If a pellet of unlysed red cells remains lying above the leucocytes, the treatment with red cell lysis buffer is repeated;

g) the leucocyte pellet is resuspended in 1-2 ml 0.9% saline;

h) the DNA is liberated by the addition of 3 ml leucocyte lysis buffer—the tube is capped and gently inverted several times, when the liquid will become viscous with DNA. The sample should be handled with care to avoid shearing and damage to the DNA;

i) proceed to DNA extraction.

4.3 Fluorideoxalate Samples

The Time 0 and 120 tubes are sent directly to the Chemical Pathology laboratory.

4.4 Urine Samples

Two aliquots are stored in 1.5 ml cryotubes (750 ul/tube yellow tops).

4.5 Logging Labelling and Storage

4.5.1 Logging and Labelling

All samples are given a unique laboratory code number and logged into the Twin Unit laboratory database. This number is used on all labels to identify all samples for a twin subject for a given visit date.

4.5.2 Storage

Those samples for immediate testing have no special storage;

Serum and urine samples which are stored at −45° C. for batched assays will be given a unique freezer location code.

4.6 Sending Samples for Assay

Appendix 1 shows the scheme for the handling/testing of blood samples.

4.6.1 Daily

The 1×500 ul routine biochemistry sample (see 5.2.1. a)) is placed in the Chemical Pathology request bag, with the 0 and 120 minute fluoride/oxalate samples. A “Twin Label” (see SOP 2) is attached to the bag, which is taken to Chemical Pathology for routine biochemistry. If sex hormone estimations are to be carried out the extra tube is included. The assays are completed on the day of the sampling, or after storage overnight. If the samples are tested next day, the fluoride/oxalate samples are spun and the clot discarded before storage.

4.6.2 Other

All other research assays are sent to other laboratories and carried out as required from the frozen serum and urine samples (see 5.3.2. b)).

4.7 Assays

The following assays are carried out.

4.7.1 Routine Biochemistry

sodium

potassium

chloride

bicarbonate

urea

creatinine

total protein

albumin

phosphate

total calcium

total bilirubin

alanine amino transferase

total alkaline phosphatase

magnesium

uric acid

4.7.1 Glucose

From fluoride/oxalate samples

4.7.2 Lipids

Measured in one aliquot after storage at −45° C.:

triglycerides

high density lipoproteins

apolipoproteins A1

apolipoproteins B

lipoprotein A

cholesterol

4.7.3 Insulin

Measured in one aliquot after storage at −45° C.

4.7.4 Sex Hormones

Measured in one aliquot:

follicle stimulating hormone (measured on the day of visit)

testosterone

Measured in one aliquot after storage at −45° C. (if required):

sex hormone-binding globulin

dehyroepiandrosterone

4.7.5 Bone Specific Markers

Measured in one aliquot after storage at −45° C.:

vitamin D binding protein

Measured in one aliquot after storage at −45° C.:

bone-specific alkaline phosphatase

4.7.6 Viitamin D Metabolites/Bone Formation Markers

Measured in one aliquot after storage at −45° C.:

1,25 (OH) vitamin D

Measured in one aliquot after storage at −45° C.:

Parathyroid Hormone (PTH)

Measured in 2-3 aliquots after storage at −45° C.:

25 (OH) vitamin D

4.7.8 Thyroid Function

TSH

FT3

FT4

4.7.9 Leptin

4.7.10 Urine

Measured in one aliquot after storage at −45° C.:

calcium

creatinine

deoxypyridinoline (Type 1 collagen crosslink)

4.7.11 Extra Tests

Extra test may be done for special protocols.

5. Use of Sample Taken from Individual Already Tested

The above description applies to the case that an individual is newly added to the database of the invention. The tests described, whether just one or any combination thereof, carried out on samples obtained from the individual are also repeatable using those samples to correct or confirm existing data or to carry out a test for the first time.

### EXAMPLE 2

### 30 Making a New Entry In or an Addition to the Database

As an alternative or addition to the protocol of Example 1, the following phenotypic data are obtained for the record of an individual on the database.

Primary

The individual is tested for information relating to the following, referred to as “primary”, phenotypes:—

Osteoporosis Related Phenotypes

Bone ultrasound

Bone density (total and regional)

Bone remodelling markers

Calcitropic hormones

Vitamin D and metabolites

Bone size

Postural stability

Fracture History

Osteoarthritis Related Phenotypes

Scores based upon x-ray (radiological, hands, knees, and hips on all twins>40 yrs)

Muscle strength

Disc Degeneration Indices (by Magnetic Resonance Imaging)

Serological markers of Inflammation

Immune Cell Subtypes (T Cell Subsets)

Immunoglobulins

Dynamic responses of immune cells to stimuli

Metabolic Syndrome/Syndrome X related phenotypes

Fasting insulin and glucose

Insulin and glucose 120 minutes post glucose load

Leptin

Lpa

HLDL, Chol, Trigs, ApoB, ApoA

Obesity (total and regional, by direct measures of adiposity)

Hypertension Related Phenotypes

Cardiac Disease (heart chamber and size and dynamics on echocardiography)

Arterial tonometry and distensibility,

Central arterial pressure, pulse wave velocity

Thrombosis/Fibrinolysis Phenotypes

Haemoglobinopathy Related Phenotypes

Airways Disease (Asthma)

Atopy/Eczema

Lung Function

IgE (specific)

Psoriasis

Acne

Skin Cancer

Moliness of Skin

Quantitative Traits Related to Cognition, Dementia, Parkinson's Disease and Intelligence

### History of Adverse Drug Reactions

### History of Substance Abuse/Addictive Behaviour

Secondary:

The individual is optionally tested for information relating to the following, referred to as “secondary”, phenotypes:—

### Lifestyle

Alcohol

Tobacco

Diet

Exercise

Comprehensive Dietary History (Validated)

### Medication History

### Family History of Disease

### EXAMPLE 3

The database of the invention can be used in the following applications.

A. Prioritisation of Candidate Genes, and Validation of High Value Drug Targets

These applications are relevant in cases where:—

several genes and/or gene regions are known which may contribute towards clinically significant risk traits; and

it is desired to prioritise one or a small number of these drug targets, and validate them.

This is achieved in the following ways.

The database including its twin resource is used to eliminate the effects of age and environment on variations in phenotypes.

The database is used to locate the gene(s) with a role in a given risk trait(s), sequence the gene(s) and identify mutations in the gene(s).

Polymorphisms with allele frequencies of at least 20% and with no complete linkage disequilibrium are selected to eliminate redundancy.

Each remaining polymorphism can be tested for association with selected phenotypes using a mean effect model.

Those phenotypes with high association with a given gene or locus can be identified—these phenotypes could be: other clinical risk traits, cell biology markers or surface receptors, circulating plasma proteins and immunoglobulins, clinical chemistry markers, circulating levels of hormones and other metabolites.

Each polymorphism can be analyzed for linkage to the candidate gene using single and multi-point linkage analyses.

The contribution of several candidate genes towards clinical risk traits, which contribute significantly to the disease can be quantified.

For the top priority gene(s), the information on the database is used for correlating genotype with clinical risk traits, and with associated biochemical and cell biology phenotypes. This gives valuable information on the targets and mechanisms of action, and the biochemical pathways.

The database is used to calculate the specificity of the candidate gene or locus, and hence the likely therapeutic index of drug candidates acting on that gene or locus, by comparing the association with clinical risk traits related to the disease, to other clinical risk traits, unrelated to the disease, but representing significant side effects.

### B. Screening and Validation of New Genotype or Phenotype Markers

These application are relevant to the case that a several new markers have been identified (such as genetic, protein or other biochemical and/or cell biological markers) and it is desired to investigate both their clinical significance and specificity. Assay methods may already be known for the markers, though it may be desired to quantify the heritability of the markers, and to prioritise and validate them, so as to decide which ones to develop.

The database of the invention can be used to determine the heritability, and prioritise and validate the markers by:

using the database, and the twin resource, to eliminate the effects of age and environment on variations in marker levels.

assaying the blood/urine samples in the database for the phenotypic marker levels, in both healthy and affected subjects.

locating the gene(s) with role in given risk trait(s), and sequencing the gene(s) and identifying mutations in the gene(s).

selecting polymorphisms with allele frequencies of at least 20%, and with no complete linkage disequilibrium to eliminate redundancy.

testing each remaining polymorphism for association with selected clinical traits and marker levels using a mean effect model.

quantifying the association of the gene (locus) with the clinical trait and marker level.

quantifying and comparing associations with other clinical traits

hence quantifying the specificity of the marker to detect the clinical trait.

In the case that there are no candidate genes, the database can be used to prioritise and validate the markers by:

assaying the blood/urine samples in the database for the marker levels, in both healthy and affected subjects.

quantifying the association of the clinical trait with the marker level and other selected phenotypes, in unaffected and affected subjects.

Thus for a given marker, the database can be used to determine its capacity and specificity to detect and quantify normal variations in healthy and affected populations for selected risk traits. A decision can then be taken as to whether and how to develop the marker(s).

### C. Accelerated and More Effective Clinical Development

### Selection of Clinical Indications for Investigation

These applications are relevant where there is a lead candidate in development, or a product on the market, which is desired to be put into clinical testing. It may be desired either to define the best clinical indication(s) or, for a selected indication, to identify patient populations which would best respond to the drug therapy. In these circumstances, the database of the invention can be used to assist in this analysis by:

using the database, and the twin resource to eliminate the effects of age and environment on variations in drug response.

constructing haplotypic profiles, with strong associations with clinical traits and biochemical phenotypes.

hence prioritising the clinical traits and the indications in which the drug is likely to be effective

defining methods for stratifying clinical trial populations for any clinical trait by haplotype and/or by phenotype.

defining selection and exclusion criteria for patient recruitment, leading to better design of clinical trials, speedier clinical trials and an ability to achieve significant results on smaller patient populations.

defining biochemical and cell biological profiles for patient selection and hence obviating the need for haplotyping, and the associated logistics, legal and ethical problems.

Selection of the Most Appropriate Dose Regimes and Drug Delivery Systems

The absorption metabolism (pharmacokinetics) and even mechanism of action (pharmacodynamics) of drugs is affected by several enzymes, and this leads to large variations in the response by patients to drug therapies. The database of the invention can help to optimise dosage regimes and dose forms by:

using the database, and the twin resource, to eliminate the effects of age and environment on variations in absorption, metabolism and mechanism of action.

sequencing the gene(s) and identifying mutations in the gene(s).

selecting polymorphisms with allele frequencies of at least 20%, and with no complete linkage disequilibrium to eliminate redundancy.

testing each remaining polymorphism for association with selected absorption, metabolic phenotypes and with associated biochemical and cell biology phenotypes using a mean effect model.

stratifying the clinical populations by high associations of metabolic/absorption and other phenotypes both with genotype and/or with associated biochemical and cell biology phenotypes.

hence allowing definition of the best dose regimes and dose forms/drug delivery systems.

### Clinical Trials

The database of the invention can be used to provide, in connection with clinical trials:

prediction on how patient populations will respond to drug therapies.

better designed phase 1 Studies—the stratification of a volunteer population by pharmacokinetics and pharmacodynamics could give far better data, and indeed more than one dose regime and dose form could be tested so as to provide the best profile of the drug for a defined patient group. It might even be worth testing more than one candidate drug.

better designed phase 2 Studies—such data can be used for phase 2 studies against comparators. Because the candidate drug, dose regimes and dose forms have been optimised during phase 1, phase 2 studies could be performed with far better exclusion criteria, would stand a far better chance of showing important differences, (important for studies with large placebo effects), and would need fewer patients recruited. This would reduce the time needed for the studies.

better designed phase 3 and phase 4 Studies—the genotyping and phenotyping results from phase 2 studies can be further refined for phase 3 studies—which are in much larger patient populations, and consume the most time and money. The benefits are the same as above, but far larger. The same applies for the design of phase 4 (post marketing), when data on even larger patient populations are available.

patients would have more appropriate and possibly individualised dosage and treatment regimes.

specific dose forms and drug delivery systems could be developed for defined patient populations.

information on responders and non-responders would minimise toxicity.

pharmacoeconomics—better data to support demands for regulatory approvals and pricing and reimbursement. (better defined patient populations, better efficacy of treatment/lower treatment costs for health authorities).

differentiating claims over competitive products.

post marketing clinical studies—as more data is available on a wider patient population, and there are more side effects, then more refined genotyping/phenotyping could define parameters so as to enable the drug to stay on the market. The database could be used to correlate data on disease parameters with data on risk traits.

### D. Epidemiological Studies

These application apply where it is desired to carry out epidemiological studies on the effects on drug therapy, vaccination or an environmental pollutant. The database of the invention can help to define the population for the design by:

using the database, and the twin resource, to eliminate the effects of age and environment.

defining clinical populations by constructing haplotypic profiles, with strong associations with defined clinical traits and biochemical phenotypes.

hence providing criteria to explain the variation in response, and define the groups most susceptible to the factor being studied.

### E. Studying Complex Diseases

During clinical studies on unselected populations, several clinically significant risk traits may be identified, and associated with the complex disease.

By using the database of the invention and associated databases covering: genomics, proteonomics, cell biology and biochemistry, it is possible to:

analyze the interaction of genes with other genes, and with proteins and other metabolites

determine genetic and non-genetic networks (e.g metabolic).

hence determine the metabolic pathways and regulatory mechanisms.

validate high value molecular targets.

### EXAMPLE 4

Samples used in connection with the database and their respective sample information are processed as follows.

Frozen samples (DNA, serum and urine, or any other clinical material) are transported from the collection centres to the database manager, using an approved courier. Samples arrive along with an electronic file and a printout of what has been sent. This should include a consignment number assigned by the collection centre, Study number (and checksum), DOB, lab reference, zygosity (in the case of twins), family number (if applicable) and volume and concentration if this is available.

Samples are logged into the database by manual or electronic entry of accompanying information. An aspect of the database is a sample tracking system, which allocates, and tracks the physical whereabouts of the samples within the database freezers. For security, each sample is stored in freezers in at least two separate buildings. Aliquots of samples may be measured, divided, diluted or concentrated by conventional means as is required for subsequent analysis. Where necessary the location of processed aliquots is allocated and tracked by the sample tracking aspect of the database.

DNA samples are subjected to any of a number of established laboratory procedures for the determination of actual or inferred DNA base sequence at regions within the human genome. The regions may be of any size (> one nucleotide) and anywhere within the genome. They are each usually defined by prior knowledge of the base sequence of a part or the whole of the region in at least one human individual.

Where the purpose of determining DNA base sequence is to discover novel/unpublished sequence in one or more human individuals, the determined sequence is entered into an aspect of the database. The method of entry and format of sequence depends on the method used for determination. The sequence is stored for reference and such further data analyses as may be required. An example of further analysis could be to identify gene coding sequence.

Where the purpose of determining DNA base sequence is to discover sequence variation between two or more chromosomes (in one or more individuals) at identical positions within the sequence, the information pertaining to the sequence variation is entered into an aspect of the database. The method of entry and format of information depends upon the method used for the determination. The sequence variation is stored for reference and such further data analyses as may be required. An example of further analysis could be to investigate the effect of the sequence variation on gene coding sequence.

Where the purpose of determining or inferring DNA base sequence is to identify and record the particular sequence variations (genotypes) in one or more individuals, the genotypes are entered into an aspect of the database. The method of entry and format of genotypes depends on the method used for the determination. The genotypes are stored for reference and such further data analyses as may be required. An example of further analysis could be the identification of an association between hypertension and an identified locus.

Whether the genetic information be a length of sequence, a particular sequence variant, or genotypes in one or more individuals, in conjunction with the phenotype information it is able to be used (in a myriad of ways) to investigate the absence or presence of correlation between human genetic variation and human phenotype variation. Any combination of genotypes and phenotypes that resides within the database can be available for analysis. Such correlations are either directly or indirectly indicative of a causal relationship between the genetic region/s and the phenotype/s, under investigation. The utility of the database is to confirm, refute, or discover such correlations.

### EXAMPLE 5

### Osteoporosis

Osteoporosis is a disease defined by low bone mass and structural deterioration of bone tissue. It leads to enhanced bone fragility and increased risk of fracture and affects 1 in 3 women and 1 in 6 men with an estimated health cost of $14 billion/annum (U.S. Figures). Calcitonin and alendronate studies indicate bone density is not the sole factor in fracture risk and that bone architecture is also important. Twin studies have shown that 60-85% of fracture risk is determined by genetic factors.

The genetic dissection of osteoporotic fracture has identified the involvement of several traits largely controlled by genes, including bone density, bone structure and muscle strength (see FIG. 1).

These risk traits operate via environmental influences to determine the probability of developing end-stage disease and ultimately bone fracture. The invention can be used to measure many of the risk factors for osteoporotic fracture as part of the standard clinical screen undertaken by many of our subjects. These include:

Bone densitometry (DEXA) at hip, lumbar spine, forearm and whole body

Spine Bone Mineral Content (BMC) & Bone Mineral Density (BMD)

Hip BMC/BMD (3 regions)

Forearm BMC/BMD

Heel ultrasound (BUA/VOS)

Personal and family history of fracture

Dietary calcium intake

Exercise history

Gynaecological, reproductive and menopausal history

HRT status

History of oral contraceptive pill use

Sex hormones

Serum/urine markers of bone turnover & metabolism

Vitamin D binding protein

25-hydroxyvitamin D

1,25-hydroxyvitamin D

Serum osteocalcin

Serum calcium

Serum phosphate

Bone-specific alkaline phosphatase

Urinary pyridinoline crosslinks

Dietary calcium absorption

Postural stability

Bone size

The genetic contribution (heritability) of many of these clinical variables has been measured using the differences between identical and non-identical twins, yielding the results below:

| Clinical Variable | Heritability |
|---|---|
| Hip intertrochanter BMD | 0.85 |
| Hip trochanter BMD | 0.83 |
| Spine BMD | 0.82 |
| Hip Wards triangle BMD | 0.70 |
| Heel Ultrasound BUA | 0.68 |
| Vitamin D binding protein | 0.59 |
| Bone-specific alkaline phosphatase | 0.41 |
| Serum calcium | 0.38 |
| Heel Ultrasound VOS | 0.34 |
| Serum osteocalcin | 0.11 |

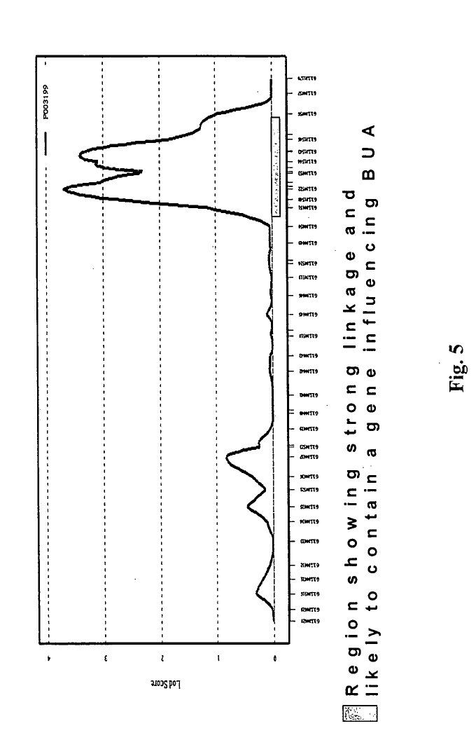

The risk factor which is discussed further in this section is highlighted (Heel Ultrasound BUA in FIG. 5). The technique consists of a simple measurement at the heel (calcaneus) derived from a quantitative ultrasound (QUS) technique. This has recently been approved in US for diagnosis of low bone mass. QUS measures 2 distinct properties of bone:

Broadband Ultrasound Attenuation (BUA) (Slope of attenuation against frequency between 200-1000 kHz)

Measures Bone Density and Structure

Correlates well with DEXA BMD at same site.

Velocity of Sound (VOS)

Measures Bone Density and Elasticity

In the general population, the distribution of BUA measurements is approximately normal in shape, characteristic of a trait controlled by several genes. The genome scan completed on BUA indicated a region on one particular chromosome showing strong evidence for linkage (see FIG. 5). This region can be subject to conventional molecular genetic strategies to identify the gene.

The database of the invention has thus been used to identify a region of a particular chromosome likely to contain a gene influencing the density and architecture of bone and hence the probability of osteoporotic fracture.

### EXAMPLE 6

### Metabolic Syndrome

Metabolic syndrome, or syndrome X, is characterised by several clinical manifestations:

Insulin Resistance

Glucose Intolerance

Hypertension

Dyslipidaemia

Type 2 Diabetes

Obesity

Underlying these outcomes are a large number of known clinical risk factors, including:

Dietary history (food frequency questionnaire, dietary composition, nutrient and calorie intake)

Anthropometric measurements

Body fat composition (total fat mass, total lean mass, central abdominal fat, thigh fat)

Fasting glucose & insulin

Insulin secretion and resistance

Glucose tolerance

Serum lipids (cholesterol, triglycerides, lipoprotein A, lipid subfractions (HDL, LDL))

Thrombosis/Haemostasis

Serum leptin

Circulating hormone levels

These risk factors are all measured as part of the standard clinical screen applied to almost all twin subjects. These variables exhibit a range of heritabilities:

| Clinical Variable | Heritability |
|---|---|
| Serum lipoprotein A | 1.00 |
| Total Fat Mass | 0.74 |
| Fasting insulin | 0.70 |
| Serum triglycerides | 0.70 |
| Insulin resistance | 0.65 |
| Body Mass Index (BMI) | 0.63 |
| Insulin secretion | 0.54 |
| Central Fat Mass | 0.51 |
| Serum Apolipoprotein B | 0.51 |
| Serum HDL | 0.49 |
| Serum cholesterol | 0.44 |
| Serum Apolipoprotein A | 0.44 |
| Serum leptin | 0.33 |

Risk factors which are discussed further in this section are highlighted. One risk factor in particular stood out in our preliminary analysis—insulin secretion. Insulin secretion is derived from a homeostasis model assessment based on fasting glucose and insulin levels and is related to the development of insulin resistance and ultimately metabolic syndrome.

The genome scan for insulin secretion yielded one region in particular which showed highly significant linkage. This region is also ready to yield to conventional molecular genetic approaches.

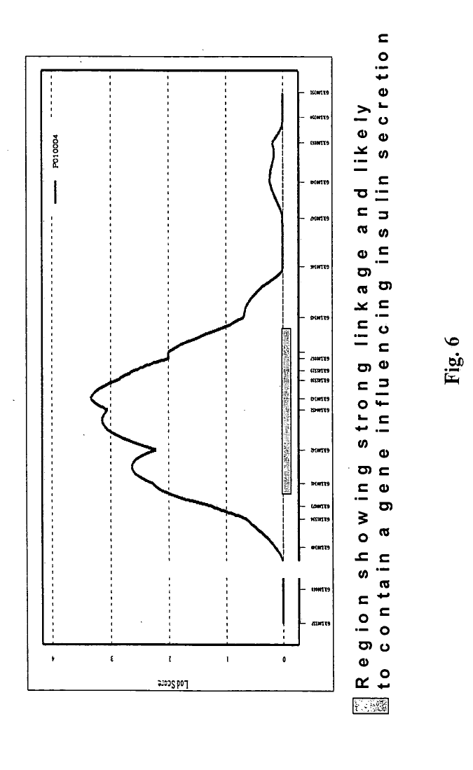

The database has been used to identify a region of a particular chromosome likely to contain a gene influencing the level of insulin secretion and hence the probability of developing metabolic syndrome (see FIG. 6).

### EXAMPLE 7

### The LPA Gene and Lipoprotein a

It is known that a gene called LPA, residing on human chromosome 6, produces a protein (Lipoprotein a or Lp(a)) that is present in the serum. The serum levels of Lp(a) are almost completely determined by variation in the LPA gene itself. Lp(a) has important clinical significance and is routinely measured as part of the standard lipid screen carried out on the twin volunteer subjects. The following clinical effects have been shown for Lp(a):

Atherogenicity (increase in level associated with increased risk of

Coronary Heart Disease)

Associated with Renal failure and proteinuria

Levels reduced in vegetarians

High serum levels correlated with progression in chronic renal failure

Implicated in hyperlipidaemic effect associated with protease inhibitors in HIV infection

The objective of this study was to rediscover the LPA gene by positional cloning.

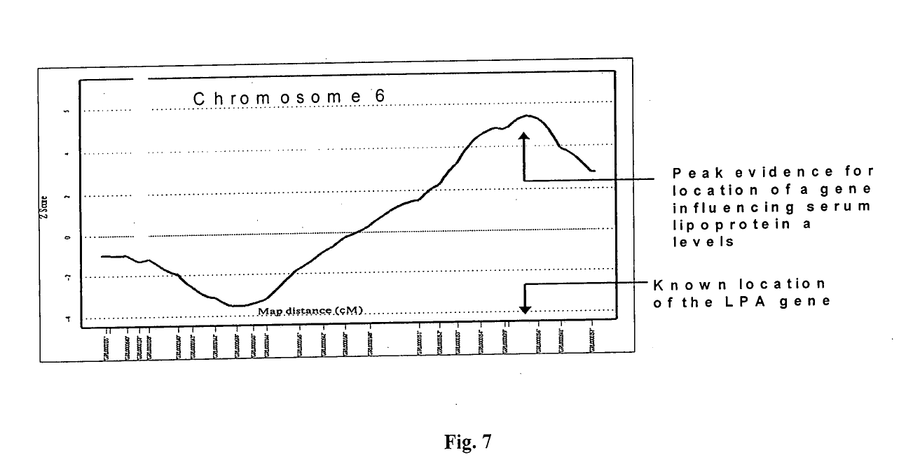

Blood samples were taken from several thousand non-identical twin pairs and the DNA extracted. A set of 400 standard markers spread across all chromosomes were tested against each DNA sample. Statistical analysis identified a relationship between serum lipoprotein a levels and the specific region of chromosome 6 known to contain the LPA gene (see FIG. 7).

This results demonstrates that the database has the ability to identify, using unselected twins, chromosomal regions containing genes with known disease involvement.

Other groups have published associations between serum lipoprotein a levels and variations in the LPA gene A large repeat polymorphism in LPA determines 40-80% of the variance in serum lipoprotein a, with the remainder being accounted for by a small number of SNPs.

This particular study is progressing to fine mapping and association which will also demonstrate the ability of the twin population to resolve the location of a susceptibility gene down to a region containing only 1 or 2 genes.

### EXAMPLE 8

### Identitifing Novel Associations with Known Genes

A detailed gene validation study was carried out on a gene that was suspected to be involved in the development of an ageing phenotype, principally osteoporosis. This association had been demonstrated in an animal model and the collaborator was particularly interested in any associations that could be discovered in humans.

The research programme was structured as follows:

1—partner provides gene(s).

2—identify common variations (e.g.polymorphisms) in the gene(s).

3—identify which variations each DNA sample contains.

4—perform statistical analysis showing relations between variations and clinical trait(s).

5—gives: disease genes validated in common human disease.

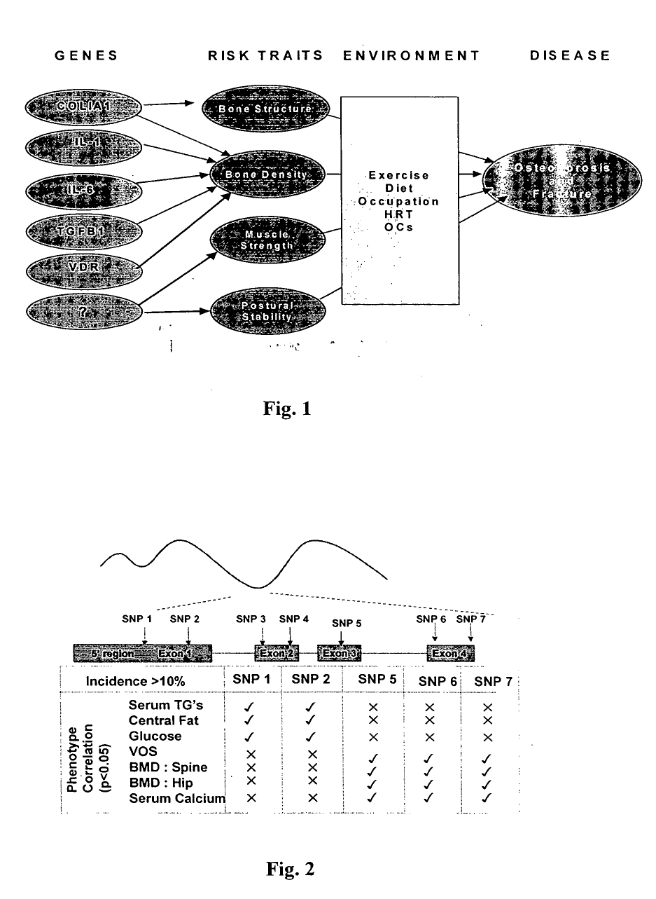

This yielded the following results (see FIG. 2), demonstrating:

Previous findings from animal studies confirmed in human disease.

A cluster of associations with a number of SNPs were observed at both ends of the gene.

SNPs at the 5′ end of the gene are implicated in metabolic syndrome.

SNPs at the 3′ end are implicated in osteoporosis.

It was possible to identify the following discoveries made using the database:

Each SNP in the gene could be used as part of a diagnostic assay.

The gene product as biotherapeutic or small molecule target.

Potential pharmacogenetic applications (e.g. patient profiling in clinical trials).

### EXAMPLE 9

### Transforming Growth Factor Beta (TGFB1)

### Identifying Novel Associations with Known Genes

TGFβ1 is a multifunctional cytokine, which regulates the proliferation and differentiation of a wide variety of cell types in vitro. TGFβ1 has been implicated in a variety of disease areas including osteoporosis, hypertension, atherosclerosis, certain forms of cancer and a number of autoimmune diseases. Consequently, the TGFβ1 gene located on chromosome 19 is an ideal candidate for investigation according to the invention, where its role in a number of different disease areas can be studied simultaneously in the same clinical population.

The invention has been operated to evaluate the role of TGFβ1 in a number of disease areas.

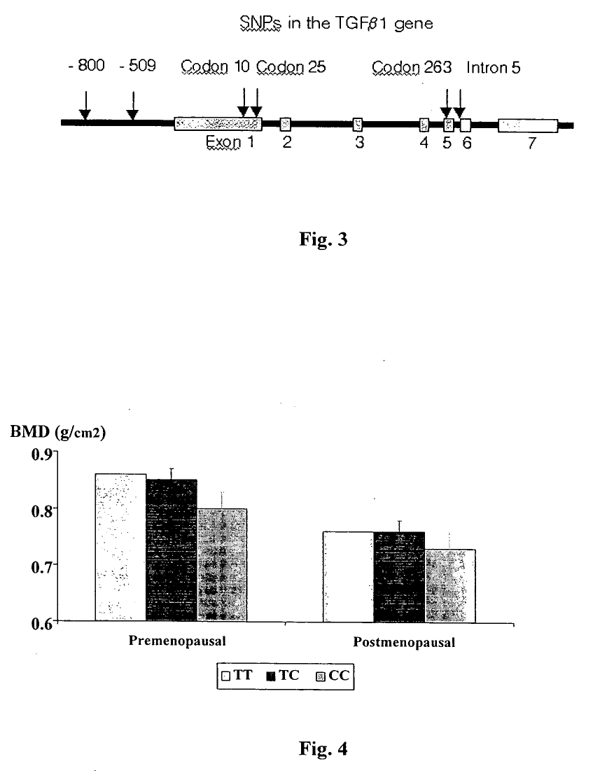

We screened the TGFα1 gene by sequencing, to identify common SNPs in the gene. We confirmed the presence of five SNPs, which have previously been reported in the gene. In addition, we also identified a novel SNP located in intron 5 of the TGFβ1 gene (see FIG. 3). The genotype of each of these six SNPs was determined in a sample of 900 non-identical twin pairs. This genotype data were analysed in conjunction with the relevant phenotype data for two disease areas, osteoporosis and hypertension.

Evidence for the involvement of TGFβ1 in osteoporosis was demonstrated by the presence of both linkage and association between the novel SNP identified in intron 5 and hip Bone Mineral Density (BMD). FIG. 4 illustrates how compared to the TT genotype, the CC genotype was associated with a 5% reduction in BMD at the femoral neck (Chi Sq=7.95. p=0.02). A similar effect was seen in both pre- and post-menopausal women, although the effect was more pronounced in the premenopausal group.

In hypertension evidence for both linkage and association was seen between blood pressure measurements and another SNP in the TGFβ1 gene located at codon 263. In this analysis the codon 263 SNP showed a significant association with both systolic (p=0.022) and diastolic (p=0.13) blood pressure. Individuals carrying the T variant of this SNP showed on average a 6 and 4 mm Hg increase in systolic and diastolic blood pressure respectively.

This study demonstrates the utility of the invention to:

identify associations between SNPs in the same gene that contribute to the variation in risk traits for different disease areas using the same clinical population; and

identify SNPs in a candidate gene (TGFβ1) related to risk traits for osteoporosis and hypertension, which could be used to assess the relative risk of an individual developing these diseases.

The invention thus provides a database containing genotype and phenotype information that can readily be used to obtain clinically and/or therapeutically and/or diagnostically useful information.

What is claimed is:

1. A database comprising a plurality of records, said records containing phenotype information and optionally sample information for an individual, wherein the record for the individual further comprises confounding information, and the sample information for the individual comprises information relating to the location of a sample of tissue or of fluid from the individual.

2. A database according to claim 1, wherein the record for an individual comprises information relating to a plurality of phenotypes and the record comprises, in respect of each phenotype:—
the phenotype observed; and
information relating to actual or potential confounding indicators in respect of phenotype.

3. A database according to claim 1, wherein said confounding information is selected from information selected from the group consisting of medication being taken by the individual, medical history, occupational information, information relating to the hobbies of the individual, diet information, family history, normal exercise routines of the individual, age and sex.

4. A database according to claim 1, wherein the phenotype and confounding information is collected at the same time from the individual.

5. A database according to claim 1, comprising a plurality of records, each record containing genotype information, and optionally sample information for an individual, wherein:
the phenotype information for the individual comprises at least one of and optionally all of osteoporosis related phenotypes, osteoarthritis related phenotypes, immune cell subtypes (such as Tcell subsets), metabolic syndrome/syndrome X related phenotypes, and hypertension related phenotypes; and
the sample information for individual comprises information relating to the location of a sample of tissue or of fluid from the individual.

6. A database according to claim 5, wherein the phenotype information further comprises at least one of and optionally all of thrombosis/fibrinolysis phenotypes, haemoglobinopathy related phenotypes and airways disease (asthma) phenotype.

7. A database according to claim 6, wherein the phenotype information further comprises information relating to one or more of the phenotypes: atopy/eczema, lung function, IgE, psoriasis, acne, skin cancer and moliness of skin.

8. A database according to claim 1 comprising a plurality of records for human individuals.

9. A database according to claim 1 wherein the sample of tissue or of fluid is selected from the group consisting of urine, serum, skin, liver, heart, bone, hair, muscle, kidney, tooth, saliva, faeces and DNA.

10. A database according to claim 1 wherein the sample information comprises the geographical location of the sample, the storage conditions of the sample and the storage reference number for reference label of the sample.

11. A database according to claim 10 wherein the sample information additionally comprises contact information enabling the individual to be contacted and retested in person.

12. A database according to claim 1, wherein each record further includes genotype information for the individual comprising one or more single nucleotide polymorphisms.

13. A database according to claim 1, comprising genotype information selected from one or more of:
(i) actual or inferred DNA base sequence at one or more regions within the genome;
(ii) a record of variation between a specified sequence on a chromosome of that individual compared to a reference sequence; and
(iii) length of a particular sequence or a particular sequence variant.

14. A method of adding information to a database according to claim 1 comprising:
(1) identifying an individual not yet included in the database;
determining phenotype information for the individual;
determining confounding information in respect of that phenotype information for the individual;
optionally determining genotype information for the individual;
optionally determining sample information for the individual that includes information relating to the location of the sample of tissue or of fluid from the individual; and
creating a record in the database to hold the phenotype, confounding and optionally genotype and/or sample information for the individual; or
(2) identifying an individual already included in a record in the database;
using sample information in the database to obtain a tissue or fluid sample for the individual;
testing the sample, thereby determining genotype or phenotype information for the individual; and
adding or confirming or amending or updating information in the record for the individual.

15. A method of identifying a correlation between phenotype information and genotype information comprising:
selecting a phenotype characteristic;
identifying a plurality of records from the database of claim 1 for individuals that comply with the selected phenotype characteristic; and
taking account of the confounding information, determining if presence of the selected phenotype characteristic is correlated with presence of any genotype characteristic in the genotype information for records in the database.

16. A method of identifying a correlation between first phenotype information and second phenotype information comprising:
selecting a first phenotype characteristic;
identifying a plurality of records in the database of claim 1 for individuals who comply with the first phenotype information;
determining if presence of the selected first phenotype is correlated with second phenotype information of records in the database.

17. A method of identifying a correlation between genotype information and genotype information comprising:
selecting a genotype characteristic;
identifying a plurality of records in the database of claim 1 for individuals who comply with the genotype characteristic;
determining if presence of the selected genotype characteristic is correlated with another characteristic of genotype information or records in the database.

18. A method of allocating priority to a candidate gene or locus, proposed as a drug target for treatment of a disease, the method comprising:—
calculating, from data on a database according to claim 1, the specificity of the candidate gene or locus for the disease;
comparing (i) the association of the disease with clinical risk traits related to the disease, to (ii) the association of the disease with other clinical risk traits unrelated to the disease, but representing significant side effects; and
hence calculating a likely therapeutic index of drug candidates acting on that gene or locus.

19. A method of analysing the relation between a genotype and a phenotype, comprising
selecting a phenotype characteristic;
identifying a plurality of records in a database according to claim 1 complying with that characteristic;
using environmental and age-related data in the database to eliminate the effects of age and environment on variations in phenotype; and
hence calculating from the database whether and if so to what extent the phenotype is correlated with a particular genotype.

20. A method of determining the capacity and specificity of a genetic marker to detect and quantify normal variations in healthy and affected populations for a selected risk trait, comprising:—
assaying samples in a database according to claim 1 for the marker levels, in both healthy and affected subjects; and
quantifying the association of the clinical trait with the marker level and other selected phenotypes, in unaffected and affected subjects.

21. A method of devising dose regimes and/or dose forms and/or drug delivery systems for a given drug in a clinical trial, comprising:—
selecting a proposed clinical population for the trial;
using data on a database according to claim 1 to stratify the clinical population by high associations of metabolism or absorption of the drug both with genotype and/or with associated biochemical and cell biology phenotypes; and
hence allowing definition of the best dose regimes and dose forms/drug delivery systems;
so as to predict and/or allow for absorption and/or metabolism of the drug by patients in the clinical population.

22. A method of predicting response to a proposed drug therapy, comprising:—
using a database according to claim 1 to select a clinical population by constructing haplotypic profiles, with strong associations with defined clinical traits and biochemical phenotypes;
using the database to eliminate the effects of age and environment in the clinical population;
hence providing criteria to predict response to the drug and variation in response to the drug, and optionally to define a sub-group of the clinical population or of the general population most susceptible to the drug being studied.

23. A method of correlating genotype and phenotype information with account taken of potential or actual confounding information, comprising use of the database of claim 1.

24. A method of diagnosing disease or predisposition to disease in an individual not showing significant signs of disease, comprising use of the database of claim 1.

25. A method of identifying a correlation between a phenotype and a genotype in a database, which comprises:
selecting a phenotype characteristic;
identifying records from the database for individuals having the selected phenotype characteristic, wherein the database comprises a plurality of records containing phenotype information, genotype information, and confounding information; and
taking account of the confounding information in the database, determining if the selected phenotype characteristic is associated with a genotype characteristic in the genotype information for records in the database, whereby the correlation between a phenotype and a genotype is identified.

26. The method of claim 25, wherein the confounding information is selected from information selected from the group consisting of medication being taken by the individual, medical history, occupational information, information relating to the hobbies of the individual, diet information, family history, normal exercise routines of the individual, age and sex.

27. The method of claim 25, wherein the phenotype information comprises one or more phenotypes selected from the group consisting of osteoporosis related phenotypes, osteoarthritis related phenotypes, immune cell subtypes, Tcell subsets, metabolic syndrome related phenotypes, syndrome X related phenotypes, and hypertension related phenotypes.

28. The method of claim 25, wherein the phenotype information comprises one or more phenotypes selected from the group consisting of thrombosis/fibrinolysis phenotypes, haemoglobinopathy related phenotypes, airway disease phenotypes, and asthma phenotypes.

29. The method of claim 25, wherein the phenotype information further comprises one or more phenotypes related to conditions selected from the group consisting of atopy/eczema, lung function, IgE, psoriasis, acne, skin cancer and moliness of skin.

30. The method of claim 25, wherein the individuals are human individuals.

31. The method of claim 25, wherein the genotype information comprises single nucleotide polymorphism information.

32. The method of claim 25, wherein the genotype information is selected from the group consisting of nucleotide sequences at one or more regions within the genome; a sequence variation; and the length of a particular sequence or a particular sequence variation.

33. The method of claim 25, wherein the database comprises records having information corresponding to twins.

34. The method of claim 33, wherein the genotype information comprises zygosity information.

35. The method of claim 25, wherein the database comprises identifiers corresponding to samples of tissue of or of fluid from the individuals.

36. The method of claim 35, wherein the samples of tissue or of fluid are selected from the group consisting of urine, serum, skin, liver, heart, bone, hair, muscle, kidney, tooth, saliva, feces and DNA.

37. The method of claim 25, wherein the plurality of records in the database contain information relating to geographical location of the sample, storage conditions of the sample, and a storage reference number of the sample.

38. The method of claim 25, wherein the plurality of records in the database contain information relating to personal contact information of individuals.
